(19) United States
(12) Patent Application Publication
(10) Pub. No.: US 2004/0122515 A1
(43) Pub. Date: Jun. 24, 2004

(54) Prosthetic valves and methods of manufacturing

(76) Inventor: Xi Chu, Mounds View, MN (US)

Correspondence Address: Xi Chu, 5273 Sunnyside Rd., Mounds View, MN, 55112, US

(21) Appl. No.: 10/717,817

(22) Filed: Nov. 20, 2003

Related U.S. Application Data
(60) Provisional application No. 60/427,747, filed on Nov. 21, 2002.
(60) Provisional application No. 60/464,384, filed on Apr. 23, 2003.

(51) Int. Cl. A61F 2/24

(52) U.S. Cl. 623/2.29; 623/2.31; 623/2.35; 623/2.42; 623/23.68; 623/901; 427/2.25; 29/890.12

(57) ABSTRACT

The present invention relates to noval design of mechanical valve prostheses and manufacturing methods. A series of prosthetic valves with novel design and a unique manufacturing approach are disclosed. These devices possess unique designs and are made of nanostructurely engineered biomaterial. In addition, a novel manufacturing approach will be used to produce these devices because the convention technique is incapable of fabricating the devices due to the small size, design requirements and material properties restrain. Furthermore, it provides the convenience and thus low cost in manufacturing. The devices are particularly but not exclusively useful in human circulation system to restore the normal functions.

### RELATED APPLICATION

The present application claims priority under 35 U.S.C. §119(e) to U.S. Ser. No. 60/427,747 filed Nov. 21, 2002, and U.S. Ser. No. 60/464,384 filed on Apr. 23, 2003, the entire contents of which is incorporated herein by reference.

### FIELD OF THE INVENTION

The present invention generally relates to mechanical valve prostheses and a method of manufacturing of the same. The valve prostheses are particularly but not exclusively directed to the use of implant in restoring normal functions in human circulation system.

### BACKGROUND OF THE INVENTION

A “valve prosthesis” is defined as an artificial device designed to replace a valvular part of the body. An existing natural valve in the body is traditionally replaced with a valve prosthesis by a surgical implantation for the treatment of valvular disease. Various designs of heart valve prostheses, in particular, have been developed to replace a defective natural heart valve.

Prior art prosthetic heart valves typically include an annular valve housing or body to provide a passageway for blood, and means attached to the annular body to open or close the blood flow passageway. These valves include valve members in the form of a single leaflet, a pair of leaflets or more leaflets. In a particular design of a heart valve prosthesis, materials used for its structure, as well as designs and fabrication contribute to the prosthesis performance and long-term operational reliability. The design of the state-of-the-art valve, as described in U.S. Pat. No. 4, 276,658 for example, has hinge areas that may hinder, and create turbulence of the blood flow. Problems of leaflet escapes at various stage of application due to shallow capture of the hinges have also been reported. Furthermore, due to the brittleness of the material commonly used in the valve structure, it is difficult to fabricate certain valve designs especially for the small diameter valves used in different parts of the body. Therefore, the needs of improved valve prostheses for various applications still remain.

Materials selected for use in prosthetic valve design have to be biocompatible and wear resistant. The conventional pyrolytic carbon is used in almost all the prior art mechanical heart valves. It is not the best biocompatible material, yet in many cases highly thrombogenic SiC has to be incorporated in the material in order to increase the hardness and wear resistance. Due to the limitations of the material used in the valve prosthesis, the patients with implanted mechanical heart valve have to take anti-coagulation drug daily through the rest of their lives to reduce clotting and thrombo embolism. Even for carefully medicate patients, various complications and side effects are widely reported. The state-of-the-art coating layer has a large material properties mismatch with the substrate resulting high residual stress and this adds risk in the application. In addition, pyrolytic carbon is extremely brittle. Due to the critical requirement of medical implants, the reliability of the materials is of extremely importance. Currently, many processing, inspection, and proof testing techniques are employed in the manufacturing of the mechanical heart valve to safeguard the product quality. The manufacturing processes are tedious and labor intensive, thereby a high cost has built into the product.

Improved designs, materials and fabrication processes are a further need in the art of valve prosthesis to achieve better efficiency and reliability. The present invention is directed to such a need.

### SUMMARY OF THE INVENTION

The present invention relates to valve prostheses, preferably a cardiac valve prosthesis, for implantation in the body. Valve prostheses of this type are usually implanted in one of the channels of the body to replace a diseased natural valve. Although the invention will be explained in connection with cardiac valve prosthesis for implantation and as a transmyocardial revascularization device, it is also possible to use valve prosthesis according to the invention in connection with implantation in other channels in the body by using the same technique as the one used for implantation of cardiac valve prosthesis. Such an implantation may, e.g., comprise the implantation of:

a. a valve in the veins,

b. a valve in the esophagus and at the stomach,

c. a valve in the cerebral fluid management,

d. a valve in the ureter and/or the vesica,

e. a valve in the lymphatic system and

f. a valve in the biliary passages and

g. a valve in the intestines

The present invention provides a series of new and improved valve prostheses. Such improved valve designs are adapted to provide a reliable and efficient performance for various applications when surgically implanted in human bodies. In particular, a more open hinge design allows a complete wash of the high area of the valve after implant and more secure hinge mechanism that increase the safety of the products. Protrusions are avoided in the blood passage to reduce the turbulence and thus reduce the destruction of the blood cells and the clotting.

The present invention also provides an improved manufacturing process. A pre-assembled manufacturing process allows the fabrication of nearly all sizes of valves for various applications.

The present invention further provides an improved composite for the fabrication of the valve prosthesis. The substrates of the valve prosthesis can be constructed from conventional materials such as metals, graphite, polymers, ceramics and paralytic carbon. An improved composite substrate can be made through molding, by mixing graphite or carbon powder with chopped carbon fibers or carbon nano fibers and organic thermosetting binders. This approach reduces the cost of material significantly. At the same time, the mechanical properties are enhanced due to the incorporation of carbon fibers in the structure.

The present invention further provides an improved pyrolytic carbon coating on the surface of prosthesis components. The process involves the integration of the pyrolytic carbon (PyC) deposition and catalytic vapor grown carbon fiber (VGCF) growth into a single operation. During the coating process, metal catalyst is continuously introduced into a fluidized bed reactor during pyrolytic carbon coating. Carbon filaments with diameters from a few to several hundred nanometers are grown catalytically in a vapor phase environment, which are referred to as vapor grown carbon fibers (VGCF). Transition metal catalyst particles play a critical role on the initiation, diameters, and growth rates of the fibers. Most importantly, the growth conditions are overlapping with those of the pyrolytic carbon deposition. Therefore, they can be co-deposited with pyrolytic carbon and reinforce the pyrolytic carbon coating. In addition to the increased mechanical strength, the biocompatibility of the improved pyrolytic carbon material will be further enhanced through the surface modification.

In the preferred embodiment of the coating material, the coating is engineered along the depth of the coating. This is achieaved by changing the coating parameters sucg temperature, gas composition and the reactor bed surface area (media size and weight). For example, the properties of the coating at the interface with the substrate are closely matched with those of the substrate so there is a minimum residual stress and good bonding. The center layer of the coating is incorporated with carbon nanofiber to gain mechanical strength and retard the crack formation and propagation. The surface layer of the valve prosthesis, however, is nanostructurely engineered in a way that all the graphitic domains are preferred aligned so that the surface is formed of the graphitic basal planes through the control of the coating parameters. The surface of the final device consists of parallel-aligned graphite basal plane domains. Therefore the activation of the blood on the device surface can be greatly alleviated or even avoided.

The present invention provides the novel designs, manufacturing method, and the integration of the re-engineered biocompatible material, which leads to improved performance of valve prosthesis. Specifically, the interaction between the blood and the valve surface is alleviated; less or no anti-coagulation will needed for the patients with the implant. The leaflet escape and fracture can be avoided and the safety of the device can be greatly enhanced.

### BRIEF DESCRIPTION OF THE DRAWINGS

The drawings form part of the present specification and are included to further demonstrate certain aspects of the present invention. The invention may be better understood by reference to one or more of these drawings in combination with the detailed description of specific embodiments presented herein. FIG. 1a. illustrates a perspective 3 dimensional sectional view of a ball valve

FIG. 1b illustrates a perspective 2 dimensional sectional view of a ball valve

FIG. 2a shows a perspective 3 dimensional sectional view of a monoleaflet valve

FIG. 2b shows a perspective 3 dimensional sectional side view of another embodiment of a monoleaflet valve housing

FIG. 2c is the sectional view of the monoleaflet valve shown in FIG. 2a

FIG. 2d illustrates perspective view of the leaflet of the valve shown in FIGS. 2a and 2c

FIG. 3a shows a perspective 3 dimensional sectional view of a open hinge monoleaflet valve (open position)

FIG. 3b shows a perspective 3 dimensional sectional view of a open hinge monoleaflet valve (close position)

FIG. 3c shows a perspective 2 dimensional sectional view of a monoleaflet valve with an open hinge structure (open position)

FIG. 3d is the leaflet of the open hinge monoleaflet valve shown in FIG. 3a and FIG. 3b

FIG. 3e is another open hinge design with a symmetric butterfly structure.

FIG. 3f is another open hinge design with a asymmetric butterfly structure.

FIG. 3g is the bottom view of another monoleaflet valve with open hinge structure and sewing rim structure

FIG. 3h is the top view of another monoleaflet valve with open hinge structure and sewing rim structure

FIG. 3i shows the relative position and top view of a monoleaflet valve shown in FIGS. 2g and 2h in transmyrocardial revascularization

FIG. 3j shows the relative position and side view of a monoleaflet valve shown in FIGS. 2g and 2h in transmyrocardial revascularization

FIG. 4a shows a perspective 3 dimensional sectional view of a open hinge bileaflet valve (open position)

FIG. 4b shows a perspective 3 dimensional sectional view of a open hinge bileaflet valve (close position)

FIG. 4c shows a perspective sectional view of the bileaflet valve with an open hinge structure (open position)

FIG. 4d shows a perspective sectional view (90 degrees with respect to FIG. 4c) of the bileaflet valve with an open hinge structure (open position)

FIG. 4e shows the perspective views of a bileaflet valve housing

FIG. 4f shows the perspective views of the leaflets of the open hinge bileaflet valve

FIG. 4g shows a perspective sectional view of the bileaflet valve with a triangular open hinge structure (open position) and a sewing rim

FIG. 4h shows a perspective sectional view (90 degrees with respect to FIG. 4c) of the bileaflet valve with a triangular open hinge structure (open position) and a sewing rim

FIG. 4i shows the flanke-like sewing rim of the triangular open hinge bileaflet valve

FIG. 4j shows a perspective sectional view of the bileaflet valve with a butterfly open hinge structure (open position) and a sewing rim

FIG. 4k shows a perspective sectional view (90 degrees with respect to FIG. 4c) of the bileaflet valve with a butterfly open hinge structure (open position) and a sewing rim

FIG. 41 shows a perspective sectional view of the bileaflet valve with an asymmetric butterfly open hinge structure (open position) and a sewing rim

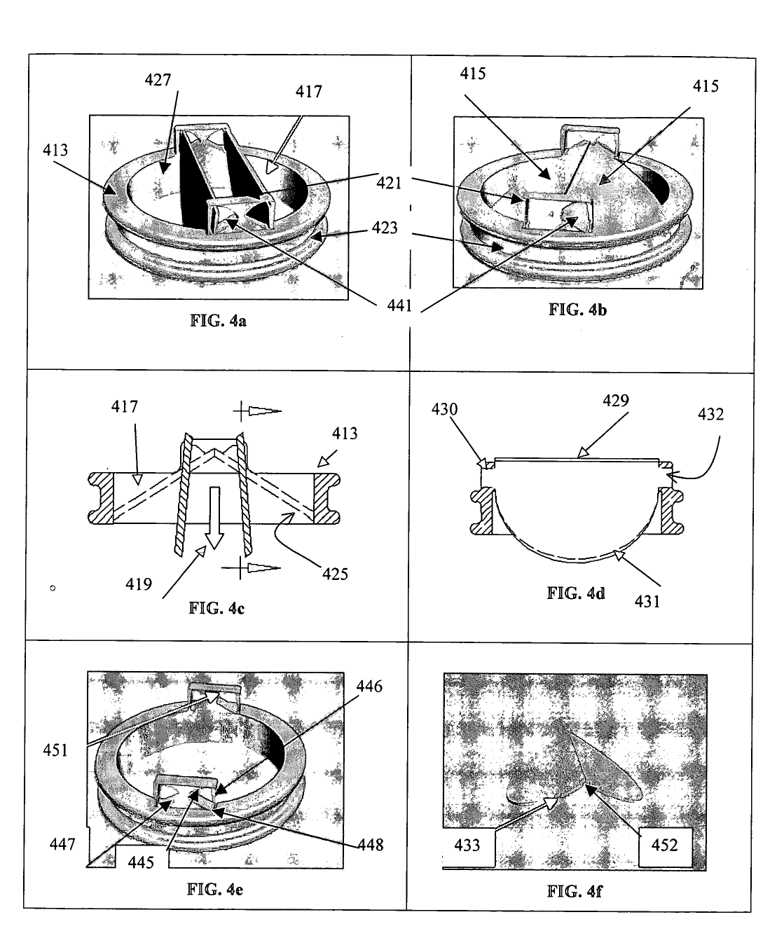

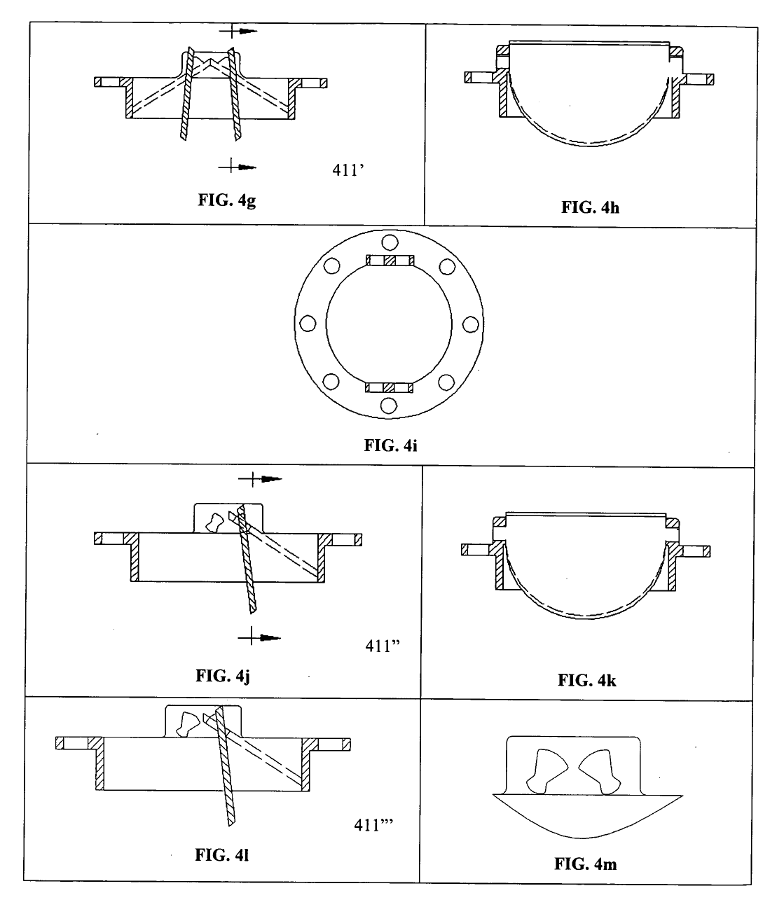

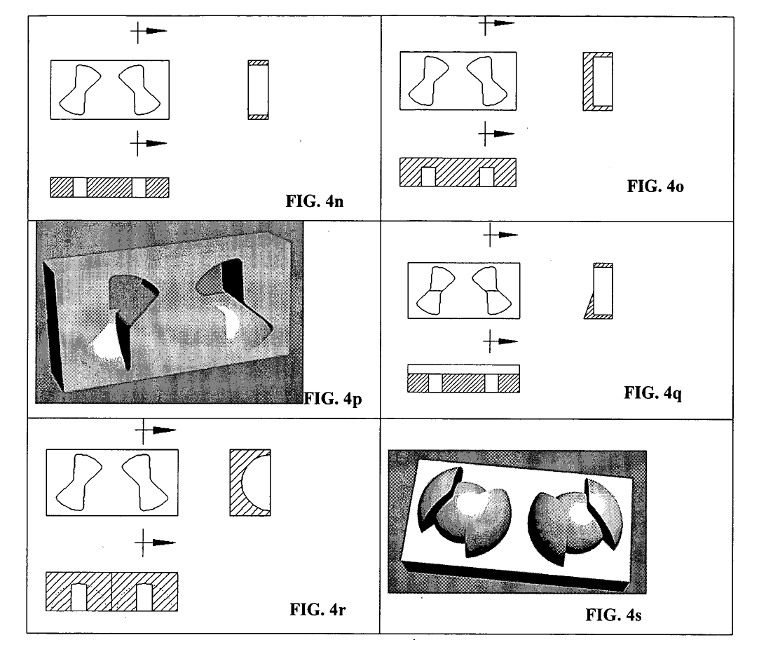

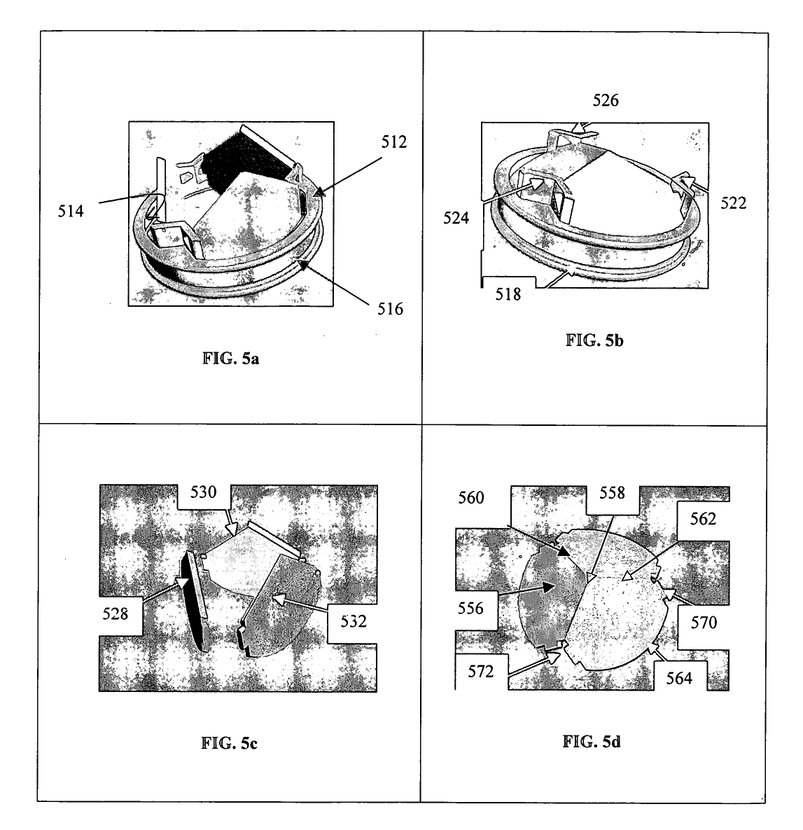

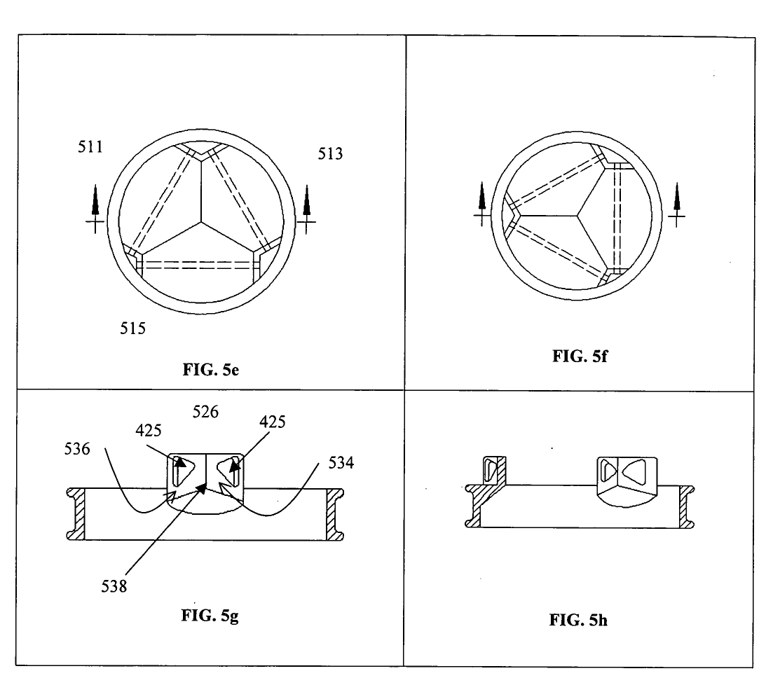

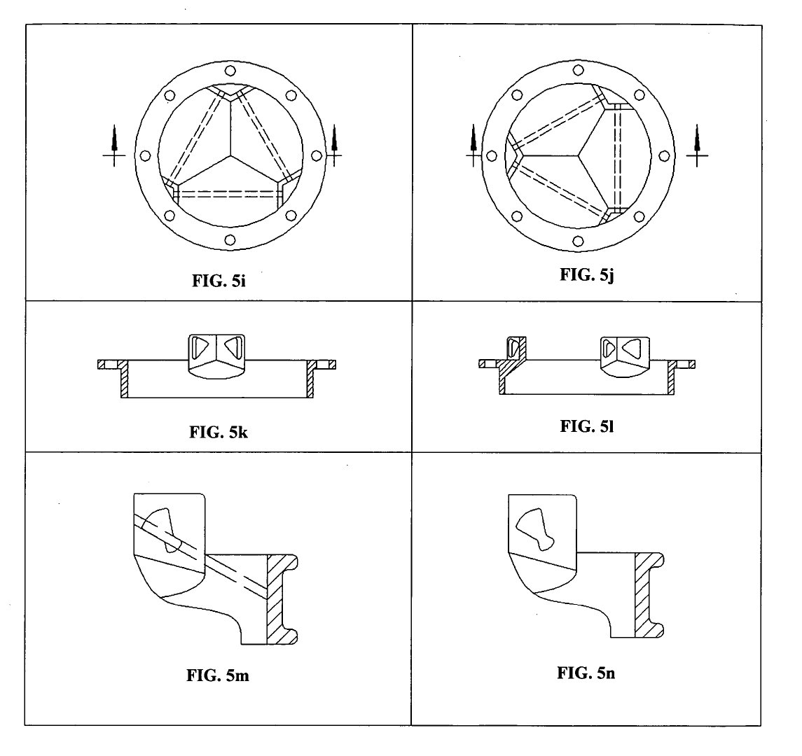

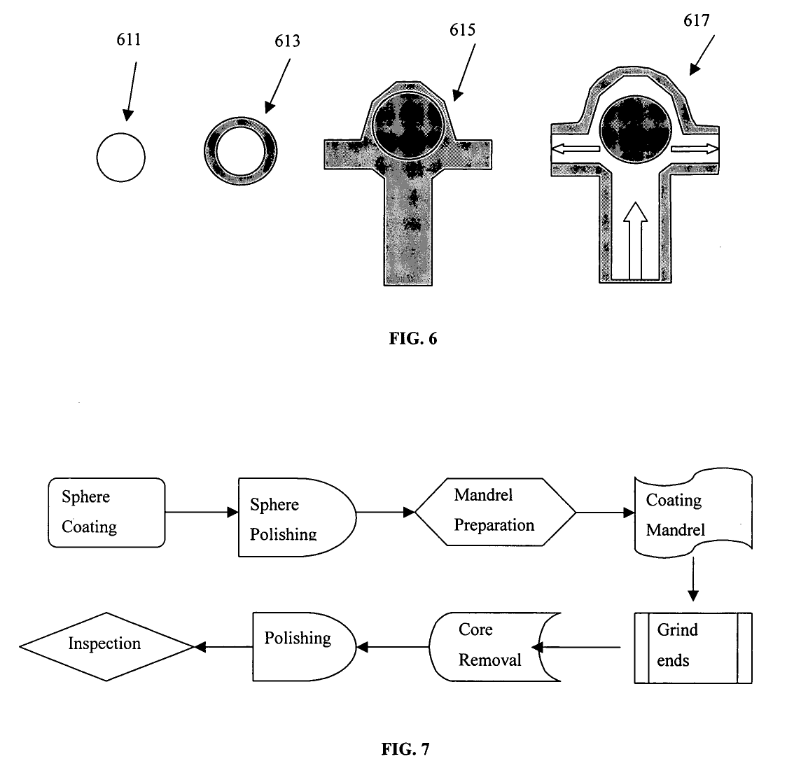

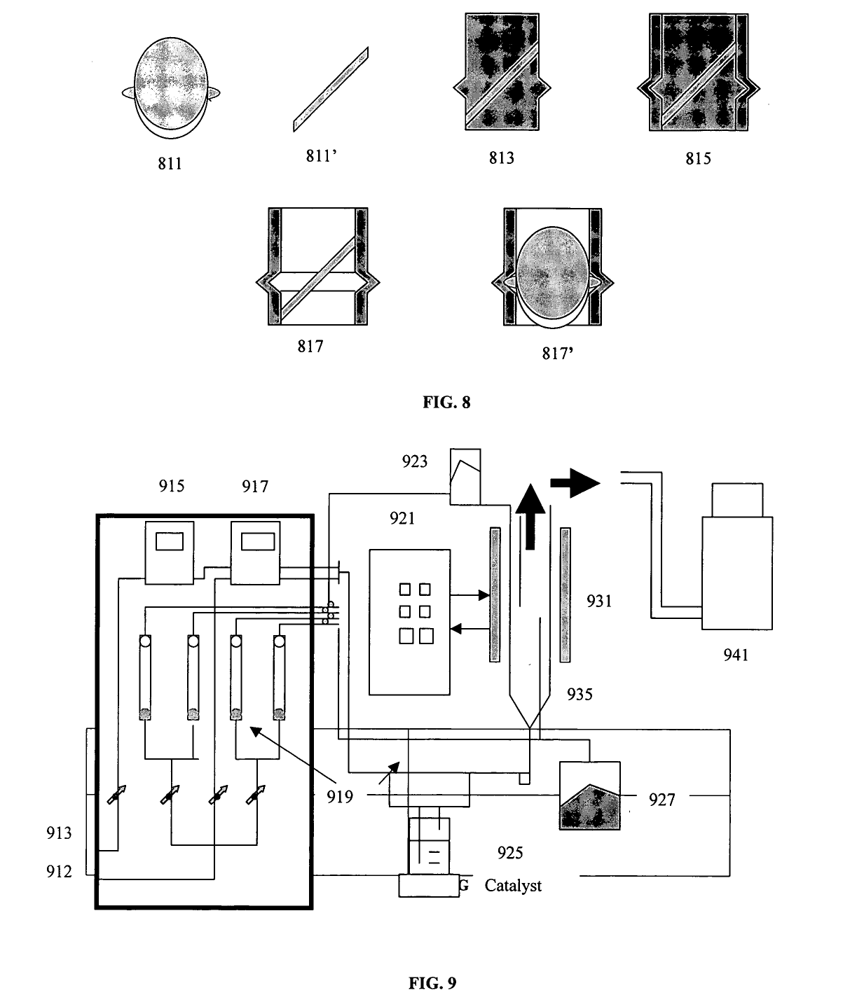

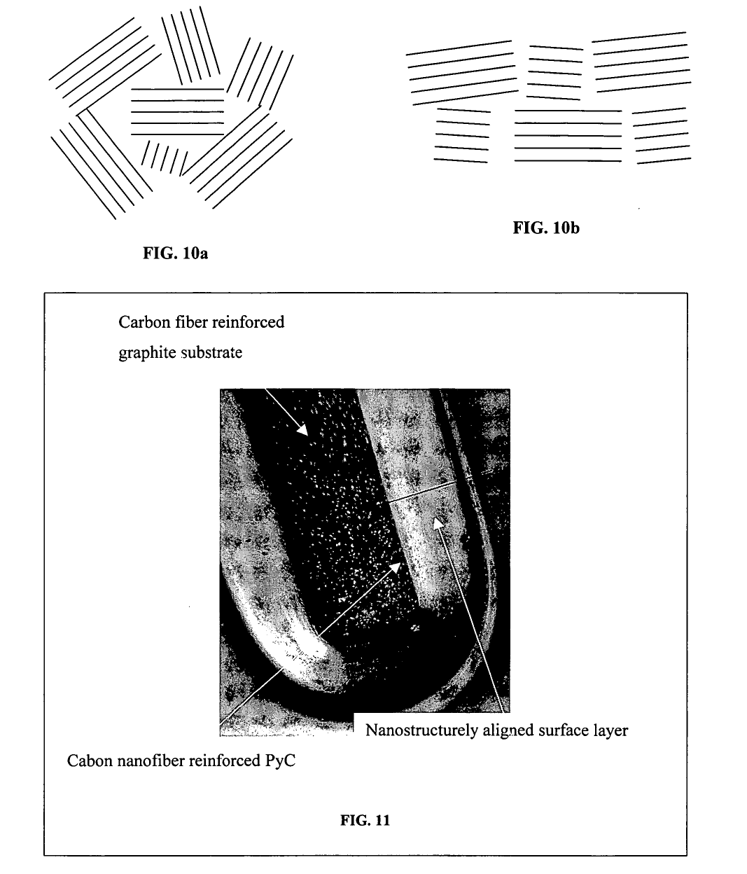

FIG. 4m shows a perspective sectional view of the hinge area of the bileaflet valve with an asymmetric butterfly shown in FIG. 41

FIG. 4n shows a perspective sectional view of the hinge area of the bileaflet valve with a symmetric open butterfly structure.

FIG. 4o shows a perspective sectional view of the hinge area of the bileaflet valve with a symmetric flat bottom butterfly structure.

FIG. 4p shows a perspective 3 dimensional view of the hinge area of the bileaflet valve with a symmetric half-open rounded bottom butterfly structure.

FIG. 4q shows a perspective sectional view of the hinge area of the bileaflet valve with a symmetric half-open butterfly structure and a flat bottom

FIG. 4r shows a perspective sectional view of the hinge area of the bileaflet valve with a symmetric closed rounded bottom butterfly structure.

FIG. 4s shows a perspective 3 dimensional view of the hinge area of the bileaflet valve with a symmetric open butterfly structure and a sphere perturtion bottom

FIG. 5a shows a perspective 3 dimensional sectional view of a open hinge trileaflet valve (open position)

FIG. 5b shows a perspective 3 dimensional sectional view of a open hinge trileaflet valve (close position)

FIG. 5c shows the structure and the relative open position of the three lealets of the trileaflet valve shown in FIG. 5a.

FIG. 5d shows the structure and the relative close position of the three lealets of the trileaflet valve shown in FIG. 5b.

FIG. 5e shows the top view of the trileaflet valve in open (dash-line of the leaflets) and close position (solid line of the leaflets)

FIG. 5f shows the top view (60 degres rotation with respect to FIG. 5e) of the trileaflet valve in open (dash-line of the leaflets)

FIG. 5g is the section view (sectional plane indicated in FIG. 5e by the two arrows) of the trileaflet valve howing the the hinge area without the leaflets

FIG. 5h is the section view (sectional plane indicated in FIG. 5f by the two arrows) of the trileaflet valve showing the the hinge area without the leaflets

FIG. 5i shows the top view of the trileaflet valve with a sewing rim in open (dash-line of the leaflets) and close position (solid line of the leaflets)

FIG. 5j shows the top view (60 degrees rotation with respect to FIG. 5e) of the trileaflet valve with a sewing rim in open (dash-line of the leaflets)

FIG. 5k is the section view (sectional plane indicated in FIG. 5i by the two arrows) of the trileaflet valve showing the the hinge area without the leaflets

FIG. 5l is the section view (sectional plane indicated in FIG. 5g by the two arrows) of the trileaflet valve showing the the hinge area without the leaflets

FIG. 5m shows a perspective sectional view of the hinge area of the trileaflet valve with a symmetric butterfly hinge structure.

FIG. 5n shows a perspective sectional view of the hinge area of the trileaflet valve with an asymmetric butterfly hinge structure.

FIG. 6 is the manufacturing process step for making small diameter ball-valve

FIG. 7 is the flow chart of valve manufacturing process

FIG. 8 is the manufacturing process step for making small diameter disc valves

FIG. 9 is the process apparatus for making the devices

FIG. 10 shows the difference between isotropic PyC and nanostrucutrely engineered PyC difference between isotropic PyC and nanostrucutrely engineered PyC

FIG. 11 The optical micrograph of the polished cross section of a leaflet structure with carbon biber reinforced graphite substrate, a carbon nanofiber reinforced inner coating layer and a nanostructurely aligned carbon outer layer

### DETAILED DESCRIPTION OF THE INVENTION

The present invention relates to a series of novel designs of valve prosthesis, with improved pyrolytic carbon biomaterial and novel fabrication processes.

Valve Prosthesis

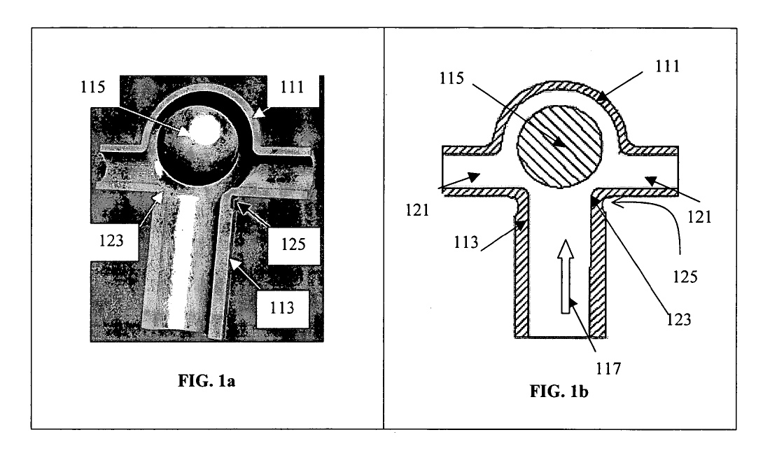

Illustrated in FIG. 1a is a sectional view of an exemplary prosthetic ball valve 111 constructed so as to embody various features of the present invention. It has an annular valve body or housing 113 which carries a valve member 115 in the form of a ball occluder which opens and closes to control the flow of blood through a central passageway 117 in the direction of the arrow 119 (FIG. 1b). The T-shaped valve body has a pair of exits 121 extends along opposite ends and perpendicular to the valve main body 113. The occluder 115 in encapsulated within a hollow space formed by the interstion between valve main body 113 and exits 121. The ball interfits with the internal wall of the exit 123 in the valve body 113, and the occluder is allowed thereby as it jumps between its open and closed positions to provide a one-way flow from the main body 113 to exits 121. While the valve 111 can operate in any orientation and is not significantly affected by gravity, for ease of explanation, the valve is shown and described with the downstream end of the valve facing upward. The passageway 117 through the valve body 113 is generally circular. The occluder 115, as best seen in FIG. 1a, is spherical with a polished surface and is generally ball-shaped. Other shape such as a disk can also be produced. The valve body 113 is formed with a peripheral groove 125 about its exterior surface that accommodates a suturing ring (not shown), which facilitates the sewing or suturing of the heart valve 111 to the heart tissue.

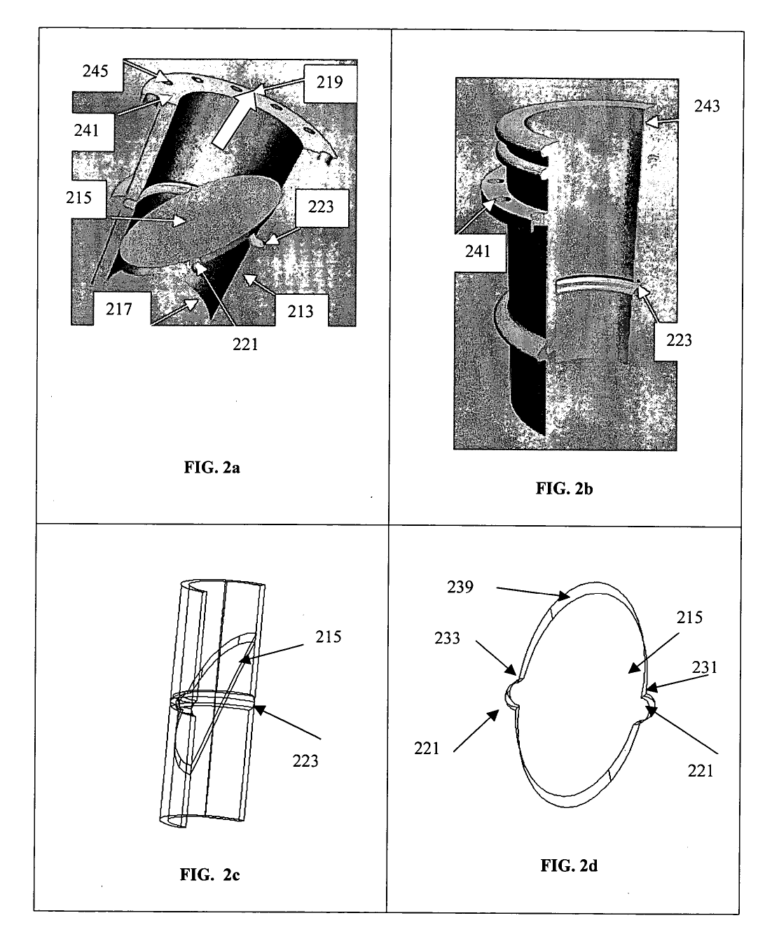

Illustrated in FIG. 2a is an examplary embodiment of a single leaflet heart valve 211 which has an annular valve body or housing 213 which carries a valve member 215 in the form of a single disc occluder which opens and closes to control the flow of blood through a central passageway 217 in the direction of the arrow 119 (FIG. 2a). A pair of ears 121 extends along opposite ends of an eccentric line across the occluder 215 (FIG. 2d) and interfits with an arcuate depression grove 223 in the valve body 213, and the occluder is guided thereby as it swings between its open and closed positions (FIGS. 2a and 2c). Meanwhile the occluder 215 can rotate freely around the center axis of the housing 213. While the valve 211 can operate in any orientation and is not significantly affected by gravity, for ease of explanation, the valve is shown and described with the downstream end of the valve facing upwnward. The valve body 213 is formed with a frang-like exit (FIG. 2a) 241 with sewing holes 245 about its exterior surface that facilitates the sewing or attachement of the valve 211 to the heart tissue. The passageway 217 through the valve body 213 is generally circular; however, a grove 223 is formed, interrupt the otherwise circular configuration of the passageway. The occluder 215, as best seen in FIG. 2d, is flat with a uniform thickness throughout and is generally disc-shaped. However, the circulary periphery is interrupted from which the ears 221 extend, leaving arcuate edge portions 231 and 233 that lie closely adjacent the arcuate portions of the interior wall in the closed position. The peripheral edge 239 of the occluder 215 is rounded in close contact with the upstream face or surface 241. The interengagement of the ears 221 and the complementary depression groove 223 serves both to retain the occluder 215 in the valve body 213 and to define the movement of the occluder therein. The ears 221, which extend at opposite ends of an eccentric line from the occluder 215 into the depression grove 223, have a generally eliipisomic configuration. The depression groove 223, with which the ears 221 inter-engage, is generally the shape of ellipsoid in cross-section and guide the ears in a generally arcuate pathway. The open position of the occluder 215, are angled from the centerline plane by about 5° to about 35°. The fully open position and fully closed position of the occluder 215 are determined by the fact that the width of the ear is relatively larger than the width of the groove to ensure the leaflet rotating between the angles without over rotation to the opposite side. In another embodiment, the grove on the valve housing can have a square cross section to guide the rotation of the leaflet and the sliding of the ears within the grove.

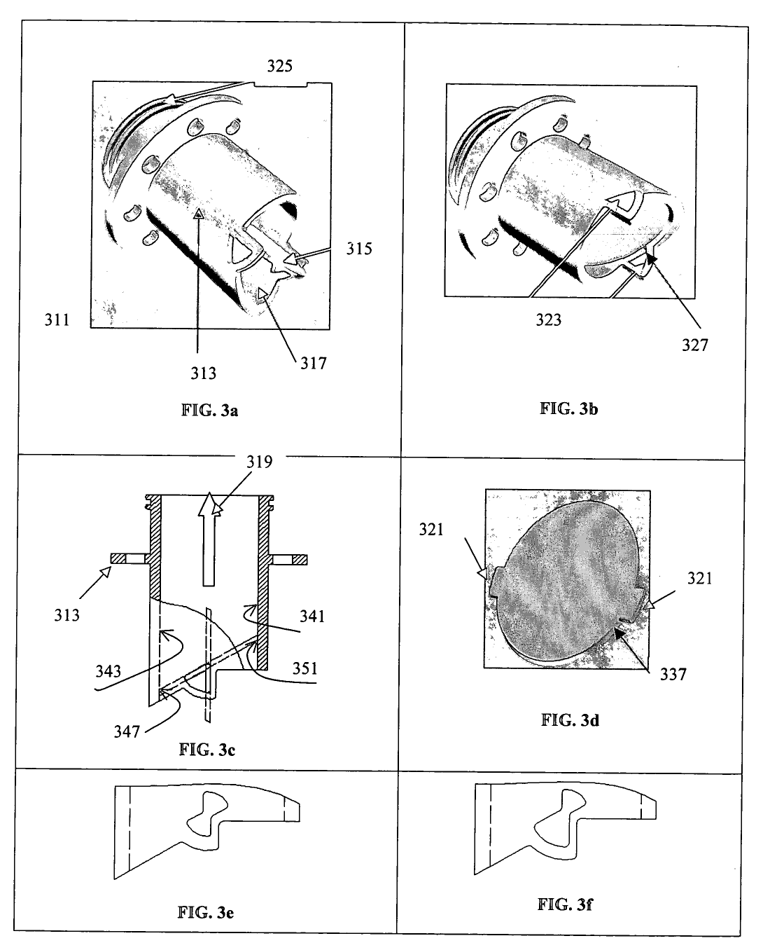

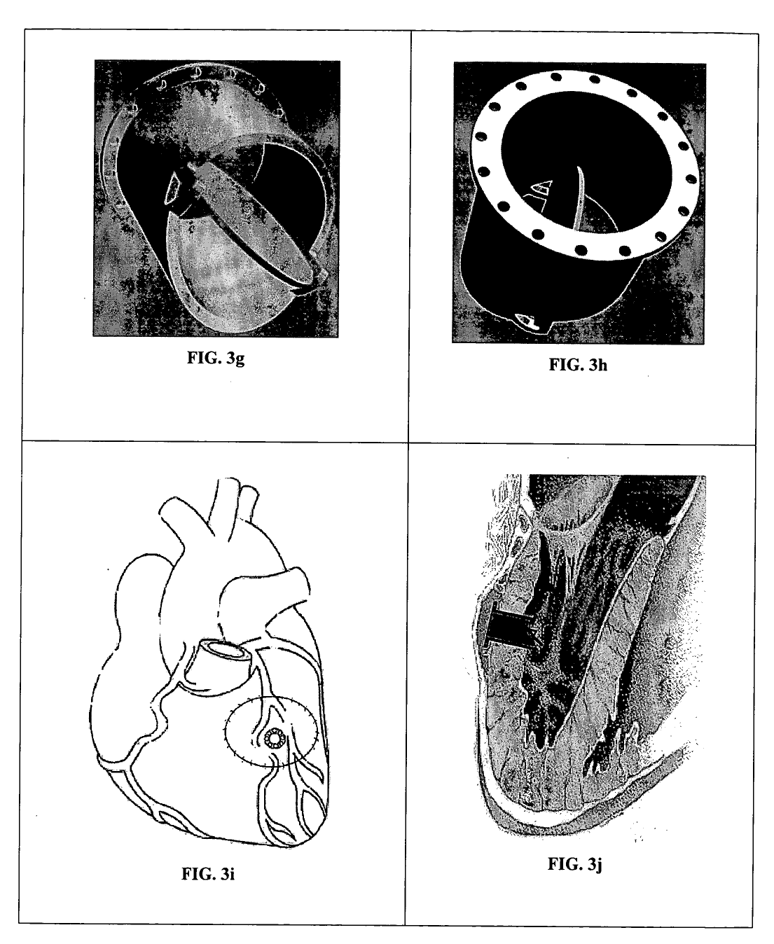

Illustrated in FIGS. 3a & 3b is an alternative embodiment of a bileaflet heart valve 311 which has an annular valve body or housing 313 which carries a valve member 315 in the form of a single disc occluder which opens and closes to control the flow of blood through a central passageway 317 in the direction of the arrow 319 (FIG. 3c). A pair of ears 321 (FIG. 3d) extends along opposite ends of an eccentric line across the occluder 315 and interfits with two hinges 323 in the valve body 313, and the occluder is guided thereby as it swings between its open and closed positions. While the valve 311 can operate in any orientation and is not signficantly affected by gravity, for ease of explanation, the valve is shown and described with the downstream end of the valve facing upward. The valve body 313 is formed with a peripheral groove 325 about its exterior surface that accommodates a suturing ring (not shown), which facilitates the sewing or suturing of the heart valve 311 to the heart tissue and to be connected to a blood vessel. The passageway 317 through the valve body 313 is generally circular; however, a pair of small diametrically opposed flat surfaces 327, in which hinges 323, interrupt the otherwise circular configuration of the passageway. The occluder 315, as best seen in FIG. 3d, is flat with a uniform thickness throughout and is generally disc-shaped. However, the circulary periphery is interrupted by straight segments 337, from which the ears 321 extend, leaving arcuate edge portions 317 that lie closely adjacent the arcuate portions of the interior wall in the closed position. The straight segments 337 are spaced apart slightly less than the distance between the opposed flat surfaces 327 of the interior wall and alternately serve as load-bearing surfaces during the swinging movement of the occluder 315. The flat surfaces 327 in the interior wall and corresponding straight segments 337 of the occluder 315 are provided so that those portions of the occluder periphery closely adjacent the centerline plane, i.e., the plane through the valve body centerline perpendicular to the flat surfaces 327, so not bind in more restricted areas of the body 313 as they move away from the centerline plane during opening. The peripheral edge 339 of the occluder 315 is rounded between its upstream face or surface 341 and its downstream face or surface 343 to eliminate sharp corners. The interengagement of the ears 321 and the hinges 323 serves both to retain the occluder 315 in the valve body 313 and to define the movement of the occluder therein. The ears 321, which extend at opposite ends of an eccentric line from the straight segments 337 of the occluder 315 into the depressions 323, have a generally rectangular configuration. The hinges 323, with which the ears 321 inter-engage, are generally the shape of arcuate troughs and guide the ears in a generally arcuate pathway. The upstream edges 351, along which the downstream edge 347 curve away from the centerline plane, along which the ears 321 lie closely adjacent in the open position of the occluder 315, are angled from the centerline plane by about 5° to about 35°. FIGS. 3e & 3f shows a flange—like outlet to allow the valve be directly attached to the tissue through sewing. This can be applied in the bi-leaflet and trileaflet valves described below in FIG. 4 and FIG. 5. Especially, when the valve is implanted in mitral and aortic position, the design can increase the effective open area (EOA) by 30 to 50% as compared with the state of the art vale using a sewing ring attachment mechanism. Shown in FIGS. 3e and 3f are two more embodiments of the hinge design. In addition, FIG. 4n-FIG. 4s show additional options for the hinges that can be applied to mono-, bi-, and tri-leaflet valves. FIG. 3i shows the relative position and top view of a monoleaflet valve shown in FIGS. 2g and 2h in transmyrocardial revascularization. FIG. 3j shows the relative position and side view of a monoleaflet valve shown in FIGS. 2g and 2h in transmyrocardial revascularization.

Illustrated in FIGS. 4a & 4b is an examplary bileaflet heart valve 411 which has an annular valve body or housing 413 which carries a pair of pivoting leaflets or valve members 415 which open and close to control the flow of blood through a central passageway 417 in the direction of the arrow 419 (FIG. 4c). The leaflets 415 are supported about eccentric axes by a pair of diametrically opposed supports 421 which extend upwardly from the annular valve body 413 as depicted in FIGS. 4a & b. It should of course be understood that the valve 411 can operate in any orientation and is not significantly affected by gravity; however, for ease of explanation, the valve 411 is shown and described with the supports 421 upstanding from the annular valve body 413. The valve body is formed with a peripheral groove 423 about its exterior surface that accommodates a suturing ring (not shown), which may be any of the various types already known in the art. The suturing ring, of course, facilitates the sewing or suturing of the heart valve 411 to the heart tissue. The passageway 417 through the valve body 413 is preferably circular and, accordingly, the internal wall surface 425 of the valve body, which defines the passage way 417, preferably has the shape of a right circular cylinder. The valve body 413 and the leaflets 415 may be made of any suitable material that is biocompatible and non-thrombogenic and that will take the wear to which it will be subjected during countless openings and closings of the leaflets. The illustrated leaflets 415 are flat and have a uniform thickness throughout, as best seen in FIGS. 4d and 4f. A minor edge 429 of the leaflet 415 is straight, and the major edge 431 is curved in a manner to match the inner surface of the passageway 417. Accordingly, the outline of the arcuate major edge 431, is generally defined by a plane cutting the right cylindrical interior wall surface 425 of the valve body 413. The minor 429 and major 431 edges of the leaflets 415 are appropriately beveled so that in the closed position of the valve 411 the major edge 431 fits against the interior wall 425 while the minor edge 429 of each of the two leaflets fits together. The upstanding supports 421 each contain a pair of generally triangular-shaped hinges 441. The material from which the valve body 413 is made has sufficient resiliency to allow the leaflet 415 to be snapped into position with the ears 432 being received in the hinges 441. To allow for freedom of movement, the radius of curvature of the vertex 448 may be slightly longer, but not more than about 3% longer, than the radius of curvature of the bottom end 433. The inner straight edge 447 may serve as a stop for the leaflet in the closed position. However, a stop is preferably provided along the edge of the leaflet itself. The outer straight edge 446 serves as a stop for the leaflet in the open position. The outer edge 446 of the hinges 441 forms an angle B of between about 5° and 10° (FIG. 4b) with a line parallel to the axis of the body 413, and the leaflets are thus stopped in an open position at an angle offset from the axis of the passageway so that back pressure will exert a force vector on the leaflets 415 to 445 close the valve 411.

The inner edge 447 of the hinge 441 forms an angle A (FIG. 4e) with a line parallel to the axis of the passageway of between 60° and 85° to allow angular movement of the leaflets 415 of between 55° and 85°. In the open position, as depicted in FIG. 4b, the main portion of the leaflet 415 has swung downward until the ears 332 abut against the outer edge 446 of the hinge 441. During the opening movement, blood flows through the valve 411 in the direction of the arrow 419. This flow, of course, occurs on the pumping stroke of the heart as a respective ventricle contracts. At the end of the stroke, the respective ventricle relaxes to draw more blood into the chamber from the atrium, and the back-pressure within the left aorta causes the leaflets 415 to swing or pivot to the closed location depicted in FIG. 4c. The proportioning of each leaflet 415 is such that it pivots about an axis which is defined by the radii of the curvature of the bottom ends 433 of the ears 432, until the cylindrical major edge surface 431 of the arcuate portion of each leaflet 415 contacts the interior side wall 425 of the passageway 417 thus sealing the outer regions of the passageway 417. At this point, the ears 432 will lie generally adjacent to the inner edge 447 of the hinge 441, and the straight minor edge portions 429 of the leaflets 415 also preferably contact each other, closing the central portion of the passageway 417 to blood flow. There is sufficient tolerance in the region of the guides and the hinges 441 to allow sealing contact along both edges of the leaflets. Because the inner straight edge 447 is not quite reached by the ears 432 before the leaflets 415 seal the valve 411, wear is reduced in this region. An interior face 451 of the supports 421 is flat and tangent to the cylindrical interior surface 425. A short straight segment 452 on the leaflets 415 between the ears 432 and the straight minor edge 429 of the leaflet 415 moves closely adjacent to the interior face 451 as the leaflets 415 pivot. This proportioning provides sufficient sealing between the leaflets 415 and the supports 421.

The above described embodiment provides excellent blood flow as the passageway 417 is cylindrical with no intrusions therein other than the smooth surfaced leaflets 415. The interior surfaces are all well washed by flowing blood. It is also of simple design and easily manufactured. The valve 411 with its guides pivoting in generally triangular hinges 441 provides well controlled movement of the leaflets with little friction, and the leaflets are free from sticking during use. The wear on the leaflets is so well distributed over the arcuate major edge 431 of the leaflet 415 as well as over the elongated ears 432 that it should not affect the working of the valve 411. As previously mentioned, the flat face 451 of the support 421 is tangent to the interior surface 425 in the preferred embodiment of the valve 411 as shown in FIGS. 4a-d to allow for unobstructed blood flow. An alternative embodiment of a valve 411′ is shown in FIGS. 4g-i wherein the up rim has sewing holes for the valve to be directly attached to the tissue. This approach will eliminate the sewing ring in a conventional vale. Therefore, the effective open area (EOA) of the valve can be increased by 30-50%. Shown in FIG. 4j and FIG. 4k is another embodiment of a valve 411″ wherein the hinge is a symmetric butterfly. Shown in FIG. 41 and FIG. 4m is another embodiment of a valve 411′ wherein the hinge is an asymmetric butterfly. Elements of the designs embodied in valves 411, 411′, 411″ and 411′″ may be rearranged in other combinations as shown in FIGS. 4n-4q. FIG. 4n shows a perspective sectional view of the hinge area of the bileaflet valve with symmetric open butterfly structure. FIG. 4o shows a perspective sectional view of the hinge area of the bileaflet valve with a symmetric flat bottom butterfly structure. FIG. 4p shows a perspective 3-D view of the hinge area of the bileaflet valve with a symmetric half-open rounded bottom butterfly structure. FIG. 4q shows a perspective sectional view of the hinge area of the bileaflet valve with a symmetric half-open butterfly structure and a flat bottom. FIG. 4r shows a perspective sectional view of the hinge area of the bileaflet valve with a symmetric closed rounded bottom butterfly structure. FIG. 4s shows a perspective 3-D view of the hinge area of the bileaflet valve with a symmetric open butterfly structure and a perturtion bottom.

FIGS. 5a & 5b illustrates an examplary tri-leaflet heart valve prosthesis generally designated 510 in open and close positions respectively. The prosthesis 510 comprises an annular valve body 512, which has a generally cylindrical inner surface 514 and an outer surface 516. Mechanical heart valves are attached to the heart with a suture ring. I have not illustrated a suture ring in connection with this description, as they are well known in this art. An up-stream edge 518 of the annular body is generally planar. A down-stream edge, on the other hand, is curved, forming three prominences equidistant from each other around the circumference of the annular body. These prominences are the locations for pivot structures 522, 524, 526 (FIG. 5c) about which leaflets 528, 530, 532 (FIG. 5b) pivot, as will be more fully described below. Each of the leaflets 528, 530, 532 is similar, and I will describe them by reference to the leaflet 528 shown in perspective view in FIGS. 5c & 5d. The leaflet 528 comprises a planar surface 556 having a central vertex 558 where all three leaflets meet in closed position (FIG. 5d). The actual mating angle will be determined by the angle chosen for the closing position of the leaflet, as shown in FIG. 5a. Adjacent the inner surface 514 of the annular body 512, the valve leaflet 528 has a curved mating edge 564. Because the leaflets are not perpendicular to the walls of the annular body when closed, the edge 564 is ellipsoid, rather than circular. Each leaflet has two ears 570-572. The ears ensure the leaflet to be captured in the triangular hinges 540, 542 and rotate along the leaflet during the open and close of the valve similar to the bileaflet valve described in FIG. 4. In the open position, the leaflets are stopped by the walls of the triangular hinges. In the close position, the leaflets are stopped by the contact of the leading edges and the curved edges at the inner surface of the valve housing.

FIGS. 5e & Sf show the valve is in close position and the dash lines 511-515 show the positions of the three leaflets when the valve is in open position. The structure of each pivot structure (FIGS. 5g & 5h) is similar and can best be understood with reference to FIGS. 3 and 4. Each pivot structure, such a pivot structure 526, has inclined walls 534, 536, which meet at a vertex 538. The vertex 538 runs parallel to the axis of the annular body 512. On each face 534, 536 there is a hinge 540, 542, which supports a portion of a leaflet as more fully described below. The hinge 540, 542 are adjacent to the vertex 538 and concave away from the vertex 538. In addition, several additional embodiments such as the sewing ring free valvse are shown in FIGS. 5i-51. FIG. 5m and FIG. 5n show two embodiments with symmetric and asymmetric butterfly hinges. The invention may be embodied in other specific forms using other himge structures shown in FIGS. 4n-4s without departing from the spirit or essential characteristics thereof. My invention therefore, is defined by the appended claims, and not by the foregoing description, and all embodiments which come with the meaning of equivalency of claims.

Manufacturing process

Shown in FIG. 6 is the manufacturing process of making a ball valve (Note FIG. 6 is a simple schematic representation only; the actual design has considered the smooth transition of the contacting surface and the space for the washing see FIG. 1). Graphite is coated with nanostructurely engineered pyrolytic carbon to form ball 613. The coated spheres are then polished through mass finishing. After polishing, each sphere is embedded in a cross-shaped graphite or carbon mandrel 615 through molding. The device is then removed from the reactor and the three ends are ground to expose the graphite core. The core is then removed through water jet because graphite is much softer than the pyrolytic carbon. A final device 617 with a hollow T-shaped pyrolytic carbon tube and a PyC coated sphere entrapped acting as a valve to allow the blood flow from the bottom to the two arms unidirectionally. The outside surface will be polished through mass finishing and the interior will be polished using abrasive slurry until a final surface finish comparable to those of the heart valve components is obtained.

FIG. 7 is the process flow chart of the ball valve manufacturing. A monoleaflet valve shown in FIG. 2 can be manufactured similarly as indicated in FIG. 8. A flat disc with two ears 811 (side view 811′) is first prepared according to design specification. The disc can be made of pure pyrolytic carbon or pyrolytic coating on thin graphite or a composite substrate described in the following section. The disc is then embedded in carbon material to form a mandrel 813 through molding. The mandrel is coated with a thick layer (more than 0.1 mm) of pyrolytic carbon or the nanostructured carbon described in the following section to form a body 815. Finally, after the removal of soft carbon in the core, a monoleaflet disc valve 817 is formed. Similarly, the polish of the surfaces and other inspection ad testing steps should be carried out.

The Substrate Preparation

In the presnt invention, substrates are prepared through molding. Graphite or carbon powder (10-80%) is mixed with commercial chopped carbon fibers or carbon nanofibers (10-80%) and organic binders (5-20%). The binder is a thermosetting polymer such as phnolic resin. The conventional molding process can be used to form the substrate green bodies. A carbonization or graphitization process in inert gas is needed to convert the green body into final substrate. The dimensional change of substrate caused by the high temperatutre treatment should be considered. The substrate is also doped with 5-10 wt % high density refractory radio opaque metals such as tungsten, W and Tantalum, Ta. These metals doping will be stable during the high temperature coating procees. The high density metal doping provides enhace image contrast during X-ray examination of valve after implantation.

Engineering the Materials at Nanometer Scales

The present invention provides a method of manufacturing nanostructually engineered biomaterials. FIG. 9 illustrates the process apparatus that makes the nanostructurely engineered biomaterials. It consists of the following well integrated sub-systems:

a. The process gas mixing and delivery system

b. The reactor hardware, heating, and control system

c. The media withdraw and the particle feeding system

d. The catalysts introducing system

e. The exhaust control and treatment system

Propane can be used as the main source of carbon for its high carbon content, low cost, availability and ease to handle. Propane line 913 (40 lbs, purity 95% with the rest of other alkanes and tracing amount of other organic compounds) was used. Nitrogen 912 was used as diluting gas. Since our process consumes a large amount of nitrogen for each run (at a flow rate of combined gas from 10 to 100 l/min.), industrial liquid nitrogen was used (99.9%, 700 lbs tank containing about 30,000 liters of nitrogen gas). Both propane and nitrogen were controlled by separate mass flow controllers 915, 917 (Davis Instrument, which control flow rate 0-50 l/min with an accuracy of 0.5% at room temperature. The mass flow controller allows the setting of the ratio of the gases and the total flow rate for each run. In addition, as shown on the panel 919, nitrogen was also used to purge the system during heating up and cooling down of the reactor, to control the meadia withdraw from the reactor during the operation, and to control (through bubbling, as will be discussed in the catalysts introducing section) and delivery catalyst to the reactor.

The system has a custom made 20 kW electrical furnaces 931 that can be operated up to 1600° C. The furnace has 12 SiC electrodes connected in series and operated at 240V AC. It allows the heating from room temperature to the reaction temperature, normally 1300° C. within 30 min. The temperature can be controlled within 1.0° C. through a digital double feedback loop controller 921.

The reactor tube 935 is made of either graphite or fussed quartz. Attempt of making ceramic reactor components was also made. The reactor tube has a diameter of 75 mm inches and a wall thickness of 2.5 mm. Its bottom is a funnel shaped with a taping angle of 40 degrees. The bottom is connected with a thin tube with an ID of 6 mm and OD of 10 mm. This thin tube is connected with processing gas line after the mass flow controllers. The small diameter inlet allows the incoming gas to create a jet within the bottom of the reactor during the reaction, therefore, moving the media and the parts within the reacting chamber of the reactor to allow the deposition of carbon on all the surfaces of the parts and media particles.

During the manufacuring process, carbon deposits on all the surfaces including the media particles and the parts. Therefore, the volume of the media increases over time. The total surface area also increases as the parts and media particles grow. To maintain the consistent process condition thus good properties, large carbon media particles were withdrawn through the side port (connected with a container in a seal system with nitrogen purge all the time) of the reactor at the bottom 927. The amount of withdraw was controlled by nitrogen pressure through solenoid valves. At the same time, small carbon particles were fed at a consistent rate of 0.5 g/min from the top feader 923 of the reactor to balance the total reactor bed material (media) volume and the surface area. The carbon media (initially loaded in the reactor) was prepared by grinding large PYC particles from the previous run and sieved to the size between 300 and 850 microns. The particles for the feeder 923 (feed into the reactor during run) were in the size range of 300-500 microns.

To co-deposit carbon nanofibers in the process, iron pentamonooxides, Fe(CO)₅ (99.5% from Aldrich) was used as the catalyst for two reasons: first, it is of low cost, and second, temperature consistent with the pyrolytic carbon process that produce high quality coating. The catalyst liquid was bubbling with nitrogen and then carried out to the process gas stream. The catalyst delivery rate of 0.1 to 4 ml/min was determined by the flow rate of the carrying gas through calibration.

During the reaction, about 50% element carbon in propane is converted into solid carbon, and the rest to carbon black and hydrocarbons. In addition, hydrogen is also a byproduct. Therefore, in the exhaust steam, there are nitrogen, hydrogen, hydrocarbons, and carbon black (soot). The exhaust was burnt before passing through a high temperature filter.

The reactor is preheated to the desired temperature with flowing N₂ (from liquid nitrogen tank). The bed materials (150 to 300 g) are ground and sieved particles from the previous runs with a size between 300-800 microns. The hydrocarbon (C₃H₈) from liquid propane tank along with diluting gas nitrogen was regulated through two mass flow controllers. The inlet pressure is maintained at 30 Psi and the amount of propane is monitored using an electronic scale. The gas mixture (the concentration was determined by experiment design) was introduced into the reactor when the reactor reaches the desired temperature. Once the run time is reached, the reaction is stopped and the reactor is cooled to room temperature and break down to extract the products. Since the density of the sample has a great impact on the mechanical strength of the mechanical properties, therefore, it was used as initial measure to monitor the process. In addition, the dimension or weight of the samples, the weight of carbon media left in the reactor (the size of the fluidized bed), the weight of the media withdrawn was measured.

The density of all the PyC samples are very close and no difference can be found between pure and carbon nanofiber reinforced PyC. However, the density of PyC related sample is about 20% higher than the state-of-the-art C—C sample and the POCO graphite (best in the class, used as substrate for our experiment). The crystalline sizes of our samples are in the range of 3 to 4 nanometers, which is in consistent with the previously reported results in the literature. Again, no difference can be found among all the PyC samples. In contrast the control C—C sample and the POCO substrates both have a crystalline size larger than a few tenth of microns.

There is a huge difference of the hardness among the samples: For the POCO graphite and the C—C control sample the hardness is low with the magnitude below 80 (Vicker). In addition, the hardness of the C—C samples varies widely from 30 to 80 due to its the porous nature. The is no remarkable difference between the hardness of pure PyC and carbon nanofiber reinforced carbon as measured by conventional indentation method for reasons described previously in the experimental section.

In addition to the mechanical strength of the coating will be greatly enhanced due to the incorporation of carbon nanofiber, the biocompatibility of the material will be further enhanced through the present invention. The interaction between the blood and the carbon surface, is highly determined by the surface structure of the carbon materials. The basic structure of carbon is the graphite-like layered structures; its basal plane is very insert to the blood while its edge sites are very active to blood cells. Depending on the process parameters, many phases of carbon such as laminar, granular, and isotropic with a wide range of properties can be produced. The low temperature isotropic pyrolytic carbon used in the state of the art mechanical heart valves is isotropic at micrometer scale with nanometer size graphitized domains randomly orientated. FIG. 10a shows the structure of isotropic PYC at a nanometer scale. It is highly interactive with the blood cells and requires the patients with mechanical heart valve implant to take blood thinner through the rest of the their lives.

In the present invention, the nanostructure engineered pyrolytic carbon are deposited on the surface layer of the heart valve components, all the graphitic domains are preferred aligned so the surface are consists of the graphitic basal planes as shown in FIG. 10b through the control of the coating parameters, that the surface of the final device are parallel aligned graphite plain. This can be done at lower gas composition, higher surface area and relative lower temperature. For example, in our process when other parameters are fixed and the process temperature is above 1300° C., propane composition is 20% or more, the formed carbon is isotropic microstructurely and surface looks rough and black. However, when the temperature is from 900 to 1200° C. and propane in diluting gas is below 20% then the carbon formed has a smoth shining metallic cluster. In the device production, coating parameters are programmed to allow a specific coating layer structure to be formed for best mechanical and biological performance.

In the preferred embodiment of the coating material, the coating is engineered along the depth of the coating. This is achieaved by changing the coating parameters such as temperature, gas composition and the reactor bed surface area (media size and weight) during the coating process. For example, the properties of the coating at the interface with the substrate are closely matched with those of the substrate so there is a minimum residual stress and good bonding. The center layer of the coating is incorporated with carbon nanofiber to gain mechanical strength and retard the crack formation and propagation. The surface layer of the valve prosthesis, however, is nanostructurely engineered in a way that all the graphitic domains are preferred aligned so that the surface is formed of the graphitic basal planes through the control of the coating parameters. The surface of the final device consists of parallel-aligned graphite basal plane domains. Therefore the activation of the blood on the device surface can be greatly alleviated or even avoided. As an example, FIG. 11 is the optical micrograph of the polished cross section of a leaflet structure with a carbon fiber reinforced graphite substrate, a carbon nanofiber reinforced inner coating layer and a nanostructurely aligned carbon outer layer.

Although the invention has been described in terms of the preferred embodiments which constitute the best mode presently known to the inventors for carrying out the invention, it should be understood that various changes and modifications as would be obvious to one having the ordinary skill in this art may be made without deviating from the scope of the invention which is defined by the claims appended hereto. More particularly, although the valve body and the leaflet are preferably made from pyrocarbon-coated graphite structures, they could be made entirely of pyrocarbon or could be made of other suitable biocompatible materials. Although the arcuate interior walls of the valve body are preferably of true circular cross-section so as to maximize the flow passageway through the valve with the leaflets in the open position, protrusions in the shape of shallow ridges could be provided at desired locations in the walls that would engage the regular edges of the leaflets; however, any such alternative arrangement should be such so that engagement occurs generally as line contact between the rectilinear edge of the leaflet and the wall of the valve body. Although flat leaflets are illustrated, the leaflets may have a simple or even complex curvature, if desired, generally as illustrated in various documentations of the U.S. patents mentioned hereinbefore.

Particular features of the invention are set forth in the claims which follow.

What is claimed is:

1. A ball prosthetic valve comprising
a T-shaped annular housing having an inner surface which defines a central passageway for the flow of a biological fluid therethrough; and
a sphere occluder entrapped in said T-shaped annular housing acting as a valve to open and close the passageway for the flow of the biological fluid unidirectionally from the main body to the two arms perpendicular to main body of the housing.

2. The ball prosthetic valve as in claim 1, wherein the T-shaped annular housing and the sphere occluder are formed of material selected from the group consisting of metal, ceramics, polymers, graphite and pyrolytic carbon.

3. The ball prosthetic valve as in claim 1, wherein the surface of the T-shaper annular housing and the surface of the sphere occluder are coated with pyrolytic carbon.

4. The ball valve as in claim 3, wherein the pyrolytic carbon is a composite comprising pyrolytic carbon, aligned nanometer sized pyrolytic carbon domains and carbon nanofibers.

5. The ball prosthetic valve as in claim 1, is implanted in a system comprising
a heart valve either an aortic, mitral, plumartumy, or tricupsular;
a valve in a transmyrocardial revascularization device;
a valve in the veins;
a valve in the esophagus and at the stomach;
a valve in the cerebral fluid management;
a valve in the ureter and/or the vesica;
a valve in the lymphatic system;
a valve in the biliary passages; and
a valve in the intestines.

6. A method of fabricating a ball prosthetic valve, comprising the steps of:
coating a graphite sphere with pyrolytic carbon to form a sphere occluder polishing the surfaces of the sphere occluder
embedding the occluder into graphite or carbon to form a T-shaped mandrel
coating the surface of the T-shaped mandrel with pyrolytic carbon
removing the graphite or carbon core
polishing all the surfaces of and the T-shaped annular housing and the sphere occluder through abrasive flow

7. A monoleaflet prosthetic valve comprising:
an annular housing having an inner surface which defines a central passageway for biological fluid flow therethrough;
a single disc occluder mounted in the annular housing acting as a valve to allow and to stop the flow of biological fluid;
a pair of ears extending along opposite ends of an eccentric line across the occluder and interfiting with a groove of the annular housing, which guide the swings of the occluder between the open and close position; and
a frang-like outlet formed on an exterior surface of the annular housing which facilitates the attachment of the valve to the tissue.

8. The monoleaflet prosthetic valve as in claim 7, wherein the annular housing and the occluder are formed of material selected from the group consisting of metal, ceramics, polymers and pyrolytic carbon.

9. The monoleaflet prosthetic valve as in claim 8, wherein the pyrolytic carbon is a composite comprising pyrolytic carbon, aligned pyrolytic carbon nanometer sized domains and carbon nanofibers.

10. A method of fabricating a monoleaflet prosthetic valve, comprising the steps of:
coating a graphite disc substrate with pyrolytic carbon to form a disc occluder machining and polishing the surfaces of the disc occluder
embedding the occluder into graphite or carbon to form a mandrel
coating the surface of the mandrel with pyrolytic carbon
removing the graphite or carbon core polishing all the surfaces of and the annular housing and the occluder through abrasive flow

11. The monoleaflet prothetic valve as in claim 7, is implanted in a system comprising
a heart valve either a arotic, mitral, plumartumy, or tricupsular;
a valve in a transmyrocardial revascularization;
a valve in the veins;
a valve in the esophagus and at the stomach;
a valve in the cerebral fluid management;
a valve in the ureter and/or the vesica;
a valve in the lymphatic system;
a valve in the biliary passages; and
a valve in the intestines.

12. A monoleaflet prosthetic valve comprising:
an annular housing having an inner surface which defines a central passageway for biological fluid flow therethrough;
a single disc occluder mounted in the annular housing acting as a valve to allow and to stop the flow of biological fluid;
a pair of ears extending along opposite ends of an eccentric line across the occluder and interfiting with two hinges in the annular housing, which guide the occluder swings between open and closed positions;
a peripheral groove formed on an exterior surface of the annular housing which facilitates the attachment of the valve to the tissue; and
a frang-like outlet formed on an exterior surface of the annular housing which facilitates the attachment of the valve to the tissue.

13. The monoleaflet prosthetic valve as in claim 12, wherein the annular housing and the occluder are formed of material selected from the group consisting of metal, ceramics, polymers and pyrolytic carbon.

14. The monoleaflet prosthetic valve as in claim 12, wherein the annular housing and the occluder are sole pyrolytic carbon or coated with pyrolytic carbon.

15. The monoleaflet prosthetic valve as in claim 13, wherein the pyrolytic carbon is a composite comprising pyrolytic carbon, aligned nanometer sized pyrolytic carbon domains and carbon nanofibers.

16. The monoleaflet prothetic valve as in claim 12, is implanted in a system comprising
a heart valve either a arotic mitral plumartumy or tricupsular;
a valve in a transmyrocardial revascularization;
a valve in the veins;
a valve in the esophagus and at the stomach;
a valve in the cerebral fluid management;
a valve in the ureter and/or the vesica;
a valve in the lymphatic system;
a valve in the biliary passages; and
a valve in the intestines.

17. A bileaflet valve prosthesis comprising
an annular valve body having a central passageway extending therethrough; and
a pair of valve leaflets and hinges supporting the pair of leaflets for substantially pivotal movement on a pair of eccentric axes between a closed position blocking the blood flow therethrough and an open position allowing blood flow therethrough, wherein the leaflets each including guides projecting in opposite directions along the pivotal axis, said guides having a rounded bottom end, said support hinges including pairs of generally triangular depression holes at generally diametrically opposite locations, each of said depressions having a curved top edge and generally straight outer and inner edges which meet at a vertex, said leaflet guides being received in said depressions with said bottom ends at the vertex thereof, said vertex being formed with a radius of curvature matched to the radius of curvature of said bottom ends and providing pivot points for each bottom end, whereby said leaflets each move between an open position wherein said guides are located generally adjacent said outer edges and a closed position wherein said guides are located generally adjacent said inner edges.

18. The bileaflet valve prosthesis as in claim 17, wherein the valve body wall which forms said central passageway is the surface of a right circular cylinder and wherein the major peripheral arcuate edge of each said leaflet is contoured so that the surface of said major edge fits flush adjacent said passageway cylindrical wall.

19. The bileaflet valve prosthesis as in claim 17, wherein a minor peripheral edge of each leaflet is straight and the edge surface thereof is planar so that the planar surfaces of said minor edges abut each other in surface-to-surface contact when said valve leaflets are in the closed position.

20. The bileaflet valve prosthesis as in claim 17, wherein the plane of each of said leaflets in the closed position makes an angle of between about 60° and about 80° with the axis of said body, and where the plane of each of said leaflets in the open position makes an angle of between about 5° and about 10° with the axis of said passageway.

21. The bileaflet valve prosthesis as in claim 17, wherein the radius of curvature of said vertex is equal to or not more than 3% longer than the radius of curvature of said bottom end.

22. The bileaflet valve prosthesis as in claim 17, wherein each of said guides has an elongated vertical edge disposed generally perpendicular to said minor edge.

23. The bileaflet valve prosthesis as in claim 17, wherein the valve body wall which forms said central passageway is the surface of a right circular cylinder and wherein at least one protrusion from said valve body wall is provided to contact each leaflet and serve as close position stop means therefor.

24. The bileaflet valve prosthesis as in claim 17, wherein upstanding supports are provided in which said hinges are formed, said supports extending inward into said passageway and having surfaces formed thereon which stop said leaflets in the open and in the closed positions.

25. The bileaflet prosthetic valve as in claim 17, wherein the annular housing and the leaflets are formed of material selected from a group consisting of metal, carbon, ceramics, polymers and pyrolytic carbon.

26. The bileaflet prosthetic valve as in claim 11, wherein the annular housing and the occluder are sole or coated with pyrolytic carbon.

27. The bileaflet prosthetic valve as in claim 17, wherein wherein the pyrolytic carbon is a composite comprising pyrolytic carbon, aligned nanometer sized pyrolytic carbon domains and carbon nanofibers.

28. The bileaflet prothetic valve as in claim 17, is implanted in a system comprising
a heart valve either a arotic, mitral, plumartumy, or tricupsular;
a valve in a transmyrocardial revascularization;
a valve in the veins;
a valve in the esophagus and at the stomach;
a valve in the cerebral fluid management;
a valve in the ureter and/or the vesica;
a valve in the lymphatic system;
a valve in the biliary passages; and
a valve in the intestines.

29. A tri-leaflet prosthetic valve comprising
an annular valve body having an inner surface and being disposed around a central axis and three substantially identical leaflets mounted in said annular valve body and configured to translate between a closed position impeding blood flow through the valve and an open position allowing blood flow therethrough, said annular body having three pairs symmetrically placed hinges spaced around an inner surface of said annular body for pivotaily supporting said leaflets, each hinge having a plane of symmetry containing said central axis; and each of said leaflets having opposed ears for engaging said hinges and a flat edge adjacent each of said ears for engaging said surface segment.

30. The tri-leaflet prosthetic valve as in claim 29, wherein each of said leaflets further comprising:
a central planar surface having a curved outer edge for engaging the inner surface of said annular body and an obliquely angled inner edge for engaging adjacent leaflets;

31. The tri-leaflet prosthetic valve as in claim 29, wherein each hinge further comprises stop means for arresting movement of the leaflets.

32. The tri-leaflet prosthetic valve as in claim 29 further comprising at least three supports for guiding said leaflets, each of said support means comprising two flat planes on said inner surface of said support.

33. The tri-leaflet prosthetic heart valve according to claim 29 wherein each of said leaflets further comprising
a central planar surface having a curved outer edge for engaging the inner surface of said annular body and an obliquely angled inner edge for engaging adjacent leaflets; and
two ears for engaging with the hinges; and two flat segment adjacent to each ear to guide the motion of the said leaflet.

34. The tri-leaflet prosthetic valve as in claim 29 wherein the valve body and the leaflet substrates are formed of material selected from the group consisting of metal, ceramics, carbon composite, and pyrolytic carbon.

35. The tri-leaflet prosthetic valve as in claim 29, wherein the valve body and the leaflets are sole pyrolytic carbon or coated with pyrolytic carbon.

36. The tri-leaflet prosthetic valve as in claim 29, wherein wherein the pyrolytic carbon is a composite comprising pyrolytic carbon, aligned nanometer sized pyrolytic carbon domains and carbon nanofibers.

37. The trileaflet prothetic valve as in claim 29, is implanted in a system comprising
a heart valve either a arotic, mitral, plumartumy, or tricupsular;
a valve in a transmyrocardial revascularization;
a valve in the veins;
a valve in the esophagus and at the stomach;
a valve in the cerebral fluid management;
a valve in the ureter and/or the vesica;
a valve in the lymphatic system;
a valve in the biliary passages; and
a valve in the intestines.

38. The prosthetic valves as in claim 12, 17 or 29, wherein the shape of the hinges is triangular, circular or butterfly.

39. The prosthetic valves as in claim 12, 17 or 29, wherein the bottom of the hinges is flat spherical depression, or spherical protrusion.

40. The prosthetic valves as in claim 12, 17 or 29, wherein the bottom of the hinges is close, open or half open.

41. Method of fabricating prosthetic valves as in claim 12, 17 or 29, comprising the steps of:
coating a leaflet substrates with pyrolytic carbon
machining and polishing the surfaces of the leaflets
embedding the leaflets into graphite or carbon to form a mandrel
coating the surface of the mandrel with pyrolytic carbon
removing the graphite or carbon core
polishing all the surfaces of and the annular housing and the leaflets through abrasive flow

42. A method of making a valve substrate comprising the steps of:
providing a mixture of graphite or carbon powder from 10 to 80 wt %, commercial chopped carbon fibers or carbon nanofibers from 10 to 80 wt % and organic thermosetting binders between 5- to 10 wt %;
doping the mixture with 5-10 wt % high density refractory radio opaque metals such as tungsten, W and Tantalum, Ta;
molding the valve substrates green body;
carbonizing the green body in inert gas at temperature higher than 1800° C.

43. A method of coating a substrate with a nanostructurally engineered pyrolytic carbon comprising the steps of:
Providing a reactor system with heating element, reactor chamber, insulation, chemical introducing and exhaust treatment systems, and particle feeding and withdrawing systems, wherein the reactor chamber contains single or multiple gas introducing ports;
Loading a pre-machined substrate in the reactor chamber filled with a media;
Introducing chemicals comprising hydrocarbons and mixtures of propane and methane, and catalysts of organic metallic compounds comprising transition metals, aloy and oxide particles;
Incorporating vapor grown carbon fibers into the high density pyrolytic carbon matrix to have randomly spaced junctions between individual fibers during in situ growth of said vapor grown carbon fibers; and
Removing the substrate from the reactor chamber.

44. The method as in claim 43, wherein the vapor grown carbon fibers are formed by flowing a gas mixture of hydrocarbons and mixtures of hydrogen and nitrogen into the reactor chamber which has been seeded with transition metal catalyst particles, wherein the catalyst can be converted from organic-metallic compounds or fine size transition metal or alloy or oxide.

45. The method as in claim 43, wherein the vapor grown carbon fibers are grown and incorporated with pyrolytic carbon by heat treatment at a temperature between 300 and 2800.degree. C.

47. The method as in claim 43, wherein the hydrocarbon comprising at least 10% by volume of said chemicals.

48. The method as in claim 43, wherein the chemicals and catalysts are introduced from the bottom ports of the reactor chamber.

49. The method as in claim 43, wherein the vapor grown carbon fibers are incorporated into pyrolytic carbon coating matrix producing a nanostructurally engineered pyrolytic carbon. In the preferred embodiment of the coating material, the properties of the coating at the interface with the substrate is closely matched with those of the substrate so there is a minimum residual stress and good bonding. The center layer of the coating is incorporated with carbon nanofiber to gain mechanical strength and retard the crack formation and propagation. The surface layer of the valve prosthesis, however, is nanostructurely engineered in a way that all the graphitic domains are preferred aligned so that the surface is formed of the graphitic basal planes through the control of the coating parameters. The surface of the final device consists of parallel-aligned graphite basal plane domains.
